(19) United States
(12) Patent Application Publication
(10) Pub. No.: US 2010/0016720 A1
(43) Pub. Date: Jan. 21, 2010

(54) ULTRASONOGRAPH

(75) Inventor: Masaki Iwasaki, Kyoto-shi (JP)

Correspondence Address: SUGHRUE MION, PLLC, 2100 PENNSYLVANIA AVENUE, N.W., SUITE 800, WASHINGTON, DC, 20037, US

(73) Assignee: SHIMADZU CORPORATION, Nakagyo-ku, Kyoto (JP)

(21) Appl. No.: 12/373,858

(22) PCT Filed: Sep. 20, 2006
(86) PCT No.: PCT/JP2006/318648

§ 371 Date: Jan. 14, 2009

(51) Int. Cl. A61B 8/14

(52) U.S. Cl. 600/443

(57) ABSTRACT

In an ultrasonograph for obtaining information relating to the inside of a subject's body using an ultrasonic wave transmitted and received by the ultrasonic probe 11 and imaging the information, after a normal imaging is finished, the ultrasonic oscillators integrated in the ultrasonic probe 11 are individually driven by the transmitter-receiver 12 to transmit and receive the ultrasonic wave, an ultrasonic image generated based on the echo signal obtained by the transmission and reception of the ultrasonic wave by each ultrasonic oscillator is analyzed by the image analyzer 18. Based on the analysis result, the progression degree of the deterioration of the probe 11 is determined and displayed on the monitor 16. Accordingly, the user can appropriately determine the probe's degree of deterioration in early stages simply by referring to the determination result.

### TECHNICAL FIELD

The present invention relates to an ultrasonograph.

### BACKGROUND ART

In an ultrasonograph, an ultrasonic probe is applied to a subject's body surface, and information relating to the inside of the subject's body is imaged based on an echo signal obtained by transmitting and receiving an ultrasonic wave by way of the probe. In such an ultrasonograph, the probe's acoustic lens may be detached or worn away due to a long-term use. An ultrasonic oscillator integrated in the probe may also be damaged because of some sort of usage problem. Since the progression of such deteriorations of the probe produces a problem such as a quality degradation of an ultrasonic image, a deteriorated probe is required to be appropriately replaced or repaired in order to maintain a stable image quality. A user can easily determine the probe's deterioration in the case where such deterioration is externally apparent or in the case where deterioration can be apparently recognized from an ultrasonic image obtained by a normal imaging. However, in the cases where there is obscure damage or where a deterioration has been occurring by small degrees over time, it is difficult for the user to evaluate the degree of deterioration.

Given these factors, Patent Document 1 discloses an ultrasonograph having the function of recording an accumulated operation time of a probe, as information regarding the probe's deterioration, in a memory integrated in the probe.

[Patent Document 1] Japanese Unexamined Patent Application Publication No. 2006-020749

### DISCLOSURE OF THE INVENTION

### Problem to be Solved by the Invention

However, the probe's operation time is merely a rough indication for deducing the degree of aging degradation, and the degree of degradation changes depending on each probe's operating conditions. Therefore, recording the probe's operation time as previously described is not enough to evaluate the probe's degree of degradation. In addition, judging an ultrasonic oscillator's damage or other state from the operation time is impossible. Given these factors, the problem to be solved by the present invention is to provide an ultrasonograph capable of accurately evaluating the ultrasonic probe's degree of degradation.

### Means for Solving the Problem

To solve the previously-described problem, the present invention provides an ultrasonograph for obtaining information relating to the inside of a subject's body using an ultrasonic wave transmitted and received by an ultrasonic probe and for imaging the information, including:

a) a transmit-receive wave controller for driving ultrasonic oscillators integrated in the ultrasonic probe so that each ultrasonic oscillator individually transmits and receives an ultrasonic wave;

b) an image creator for creating an ultrasonic image based on an echo signal obtained by the transmission and reception of the ultrasonic wave by each ultrasonic oscillator;

c) an image analyzer for performing a predetermined analysis of the ultrasonic image;

d) a determiner for determining a deterioration degree of the ultrasonic probe based on an analysis result by the image analyzer; and

e) a determination result displayer for displaying a determination result on a monitor.

The ultrasonograph according to the present invention may preferably further include:

f) a characteristic value memory unit for memorizing a characteristic value of the ultrasonic image obtained in an analysis by the image analyzer; and

g) a secular change displayer for creating a graph illustrating a secular change of the characteristic value and for displaying the graph on the monitor.

The image analyzer in the ultrasonograph according to the present invention may have the function of obtaining the width of a stripe appearing in the ultrasonic image due to a multiple reflection of an ultrasonic wave in the ultrasonic probe. In this case, the determiner determines, based on the stripes' interval, the progression degree of the abrasion of an acoustic lens provided in the ultrasonic probe.

In addition, the image analyzer may have the function of obtaining the sum of the brightness values in each line in the ultrasonic image. In this case, the determiner determines whether or not the ultrasonic oscillator is damaged based on the sum of the brightness values obtained for each line.

In the present invention, a “line” in an ultrasonic image is a line of picture elements in the image each corresponding to an echo signal obtained by each ultrasonic oscillator. The brightness change in the line reflects the temporal change of an echo signal intensity obtained by each ultrasonic oscillator.

Furthermore, the image analyzer may have the function of determining, in the case where a sum brightness for the entire ultrasonic image is equal to or less than a predetermined value, whether or not a brightness distribution of each line in the ultrasonic image satisfies a predetermined pattern. In this case, the determiner determines the presence or absence of a detachment of an acoustic lens provided in the ultrasonic probe or the progression degree of the detachment, based on the presence or absence of a line satisfying the predetermined pattern or the occurrence ratio of such lines.

### EFFECTS OF THE INVENTION

With the ultrasonograph according to the present invention having the aforementioned configuration, the user can easily know whether or not the ultrasonic probe is required to be repaired or replaced simply by referring to the determination result displayed on the monitor. Accordingly, it is possible to prevent a wrong diagnosis, etc, caused by taking an ultrasonic image with a deteriorated probe, which contributes to the development of the diagnostic capability.

In the case where the ultrasonograph according to the present invention has the function of displaying the secular change of the characteristic value, the user can comprehend the ultrasonic probe's deterioration in earlier stages by referring to the characteristic value's secular change displayed on the monitor. Therefore, it is possible to estimate an appropriate replacement time.

### BRIEF DESCRIPTION OF THE DRAWINGS

FIG. 1 is a block diagram illustrating a main portion's configuration of the ultrasonograph according to an embodiment of the present invention.

FIG. 2 is a diagram illustrating an example of an echo signal obtained in executing a probe deterioration determination scan mode in the ultrasonograph of the same embodiment.

FIG. 3 is a graphic illustrating an example of an ultrasonic image generated in executing a probe deterioration determination scan mode in the ultrasonograph of the same embodiment: (a) is an image in the case where a probe is normal, (b) is an image in the case where some of the ultrasonic probes are damaged, and (c) is an image in the case where an acoustic lens is detached.

FIG. 4 is a diagram illustrating a characteristic value's secular change display window in the ultrasonograph of the same embodiment.

### EXPLANATION OF NUMERALS

11 . . . Ultrasonic Probe
12 . . . Transmitter-Receiver
13 . . . Beam Former
14 . . . Signal Processor
15 . . . Digital Scan Converter (DSC)
16 . . . Monitor
17 . . . Image Capturing Unit
18 . . . Image Analyzer
19 . . . Analysis Result Memory Unit
20 . . . Controller
21 . . . Input Unit

### BEST MODE FOR CARRYING OUT THE INVENTION

Hereinafter, the best mode for carrying out the present invention is described using an embodiment.

### Embodiment

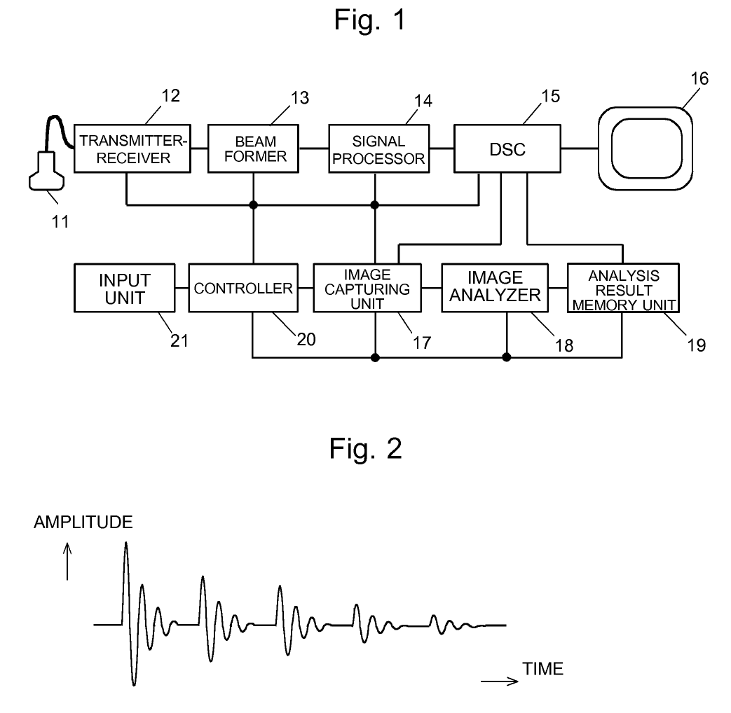

FIG. 1 is a block diagram illustrating a main portion's configuration of the ultrasonograph according to the present embodiment. In an ultrasonic probe 11, a plurality of ultrasonic oscillators are integrated. The application timing of a drive pulse to each oscillator, the receiving timing of a reflected wave by each oscillator, etc are appropriately controlled by a transmitter-receiver 12. At this point in time, the transmission and reception of the ultrasonic wave is appropriately controlled in accordance with the probe used in order to perform an ultrasonic scan in a variety of scan modes such as a liner scan, convex scan, or sector scan. The echo signal received by the ultrasonic probe 11 is delivered to a beam former 13 via the transmitter-receiver 12, and then phased and summed in the beam former 13. Consequently, an echo signal received by each ultrasonic oscillator is synthesized into a single beam signal. The beam signal is fed to a signal processor 14, and processed by a gain adjustment process, logarithmic compression process, detection process, or other processes. Additionally, the beam signal is processed by a coordinate transformation and interpolation process in a digital scan converter (which will hereinafter be called a “DSC”) 15 to create an ultrasonic image signal. The ultrasonic image signal is sequentially read out from an image memory provided in the DSC 15 and displayed on the monitor 16. The operation of each unit is controlled by a controller 20 including a central processing unit (CPU), and the user's instruction is delivered to the controller 20 through an input unit 21 including a keyboard, a variety of operation buttons, trackball, or other units.

The aforementioned configuration is the same as in a normal ultrasonograph. However, the ultrasonograph according to the present invention has, in addition to the aforementioned configuration, an image capturing unit 17 for capturing an image provided from the DSC 15, an image analyzer 18 for performing a predetermined image analysis based on the image captured by the image capturing unit 17, and an analysis result memory unit 19 for recording the result of the image analysis. Although these units may be realized by dedicated hardware components, they may be implemented as software components by installing a predetermined program (which will be hereinafter called a “probe deterioration determination program”) on a memory unit (not shown) provided in the ultrasonograph for example.

Hereinafter, an operation in performing a probe deterioration determination in the ultrasonograph according to the present embodiment will be described. In the ultrasonograph according to the present embodiment, after an imaging by a normal imaging program is finished, the user performs a predetermined operation through the input unit 21 to terminate the imaging program. Simultaneously, the probe deterioration determination program is activated, and an ultrasonic scan in a probe deterioration determination scan mode, generation of an ultrasonic image based on the echo signal obtained by the ultrasonic scan, and the probe's deterioration determination by the analysis of the image are automatically executed.

In the normal imaging as previously described, a plurality of ultrasonic oscillators are simultaneously (or with a delay time) driven, and the echo signal obtained by each ultrasonic oscillator is summed by the beam former 13 to create a single piece of beam data. On the other hand, in the probe deterioration determination scan mode, an ultrasonic wave is sequentially transmitted and received by each of the ultrasonic oscillators integrated in the probe 11. In addition, in the normal imaging, the beam data is processed by a coordinate transformation and interpolation process in the DSC 15; however, in the probe deterioration determination scan mode, the coordinate transformation and interpolation process are not performed. Instead, regardless of the ultrasonic oscillators' interval, an ultrasonic image is formed so that an echo signal transmitted from one ultrasonic oscillator corresponds to a picture element line (which will be hereinafter called a “line”) with a one-dot width. Hence, even in the case where a convex probe or sector probe is used, a rectangular image is formed as in the case where a linear probe is used. The ultrasonic image generated in this manner is captured by the image capturing unit 17 and sent to the image analyzer 18, where a predetermined analysis is performed.

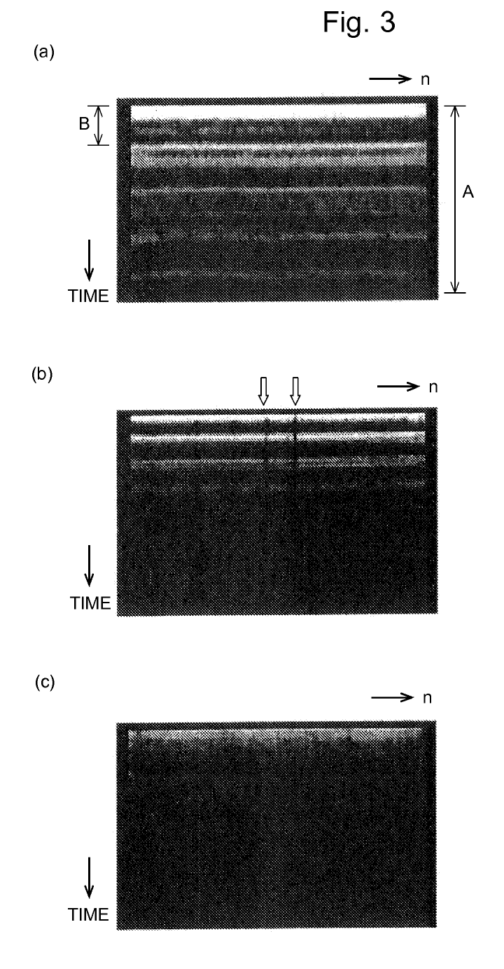

The determination of the probe's deterioration in the ultrasonograph according to the present embodiment is performed in the state where the probe 11 is being held in a probe holder provided in the ultrasonograph after a normal imaging is finished. Hence, in performing the probe deterioration determination scan mode, an ultrasonic wave is transmitted and received with the ultrasonic wave emitting surface (i.e. the surface applied to the subject's body in a normal imaging) of the probe 11 directed towards the air. Since an acoustic lens and air have significantly different acoustic impedance, most of the ultrasonic waves transmitted from the ultrasonic oscillator in such a state reflect at the boundary between the acoustic lens and the air, and multiply reflect inside the probe 11. An example of the echo signal transmitted from each ultrasonic oscillator to the transmitter-receiver 12 at this point in time is illustrated in FIG. 2. Additionally, an example of the ultrasonic image generated in the DSC 15 based on the echo signal is illustrated in FIG. 3(a). The vertical axis of the ultrasonic image corresponds to the time axis in FIG. 2, and the horizontal axis thereof corresponds to the serial number n for each ultrasonic oscillator. In the probe deterioration determination scan mode as previously described, an ultrasonic wave multiply reflected in the probe 11 is projected to the ultrasonic oscillator at specific time intervals which are dependent on the acoustic lens' thickness. Consequently, a plurality of stripes appear on the image.

Next, the following analysis is performed in the image analyzer 18, based on the ultrasonic image obtained as previously described.

### (1) Determination of the Failure of the Ultrasonic Oscillators

In the case where some of the ultrasonic oscillators among a number of those integrated in the ultrasonic probe 11 are damaged, as in the image illustrated in FIG. 3(b), the line or lines corresponding to such damaged oscillators (the portions indicated with the bold arrows in the figure) characteristically become entirely dark. Therefore, the sum of the brightness values for each line is first obtained by the image analyzer 18, and then the sum value of the brightness values for each line is compared with the sum value of the brightness in the brightest line. In the case where the former sum value does not reach a predetermined level, it is determined that the ultrasonic oscillator corresponding to the line is damaged.

### (2) Determination of the Detachment of an Acoustic Lens

In the case where an acoustic lens is detached, as in the image illustrated in FIG. 3(c), only the first few picture elements are characteristically bright, and the picture elements other than those are dark. Hence, the sum of the brightness in the image is calculated by the image analyzer 18, and in the case where the sum value does not reach a predetermined level, the sum value of the brightness for the first half of each line and the sum value for the last half are further obtained, and it is determined whether or not each of them exceeds a separately set threshold value. At this point in time, in the case where only the first half of a line exceeds the threshold value, it is determined that the line fulfills the aforementioned characteristic, which leads to the conclusion that the lens is detached. The progression degree of the detachment may be determined in accordance with the occurrence ratio of such lines. In the case where both the first half and last half of the line are below the threshold value, it is determined that the ultrasonic oscillator corresponding to the line has a defect as in the previously described case.

### (3) Determination of the Abrasion of an Acoustic Lens

As described earlier, the width of the stripe caused by a multiple reflection in an ultrasonic image is dependent on the acoustic lens' thickness, and the width of the stripe characteristically becomes small as the acoustic lens becomes worn. Therefore, the width of the stripe may be obtained by performing a fast Fourier transform (FFT) analysis or other analysis to the ultrasonic image, and in the case where the stripe's width is equal to or less than a predetermined value, it is determined that the abrasion of the acoustic lens is progressing and the replacement of the probe is required.

The aforementioned determination result is displayed on the monitor 16. Hence the user can easily know whether or not the ultrasonic probe 11 is required to be repaired or replaced simply by referring to the determination result.

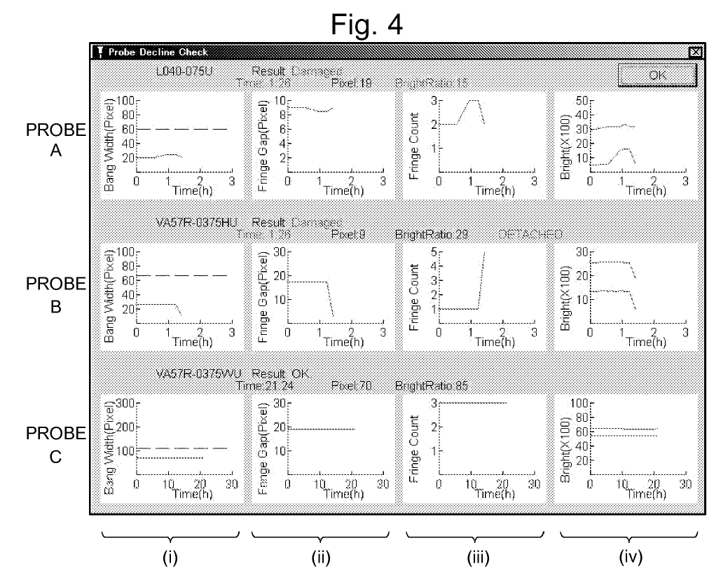

A variety of characteristic values obtained by the image analyzer 18 are memorized in the analysis result memory unit 19. When the ultrasonograph is activated the following time, the secular change of the characteristic values from the point in time when the ultrasonograph was used for the first time until the point in time it was used the last time are displayed on the monitor 16. FIG. 4 is an example of such a display window illustrating the characteristic values' secular change. In this example, the secular changes are illustrated for three ultrasonic probes A through C, each for (i) the main bang width, (ii) the stripe width, (iii) the number of stripes, and (iv) the sum value of brightness in the brightest line and that in the darkest line. The main bang width signifies the width of the entire stripe pattern caused by an ultrasonic wave's multiple reflection (A in FIG. 3(a)). The number of stripes in the image is calculated by dividing the main bang width by the stripe width (B in FIG. 3(a)). By referring to the characteristic values' secular change, the user can comprehend the deterioration of the ultrasonic probe in earlier stages and estimate an appropriate replacement time.

As previously described, the best mode for carrying out the present invention has been explained by using an embodiment. It should be noted that the present invention is not limited to the aforementioned embodiment, but an appropriate change can be allowed within the scope of the present invention. For example, in the aforementioned embodiment, when a normal imaging program is terminated by the user, the probe deterioration determination program is automatically activated and a series of probe deterioration determination operations are performed from transmitting and receiving the ultrasonic wave in the probe deterioration determination scan mode to displaying the determination result. Other than this, the determination for the probe's deterioration may be appropriately performed in response to the user's instruction through the input unit.

1. An ultrasonograph for obtaining information relating to an inside of a subject's body using an ultrasonic wave transmitted and received by an ultrasonic probe and for imaging the information, comprising:
a) a transmit-receive wave controller for driving ultrasonic oscillators integrated in the ultrasonic probe so that each ultrasonic oscillator individually transmits and receives an ultrasonic wave;
b) an image creator for creating an ultrasonic image based on an echo signal obtained by transmission and reception of the ultrasonic wave by each ultrasonic oscillator;
c) an image analyzer for performing a predetermined analysis of the ultrasonic image;
d) a determiner for determining a deterioration degree of the ultrasonic probe based on an analysis result by the image analyzer; and
e) a determination result displayer for displaying a determination result on a monitor.

2. The ultrasonograph according to claim 1, further comprising:
f) a characteristic value memory unit for memorizing a characteristic value of the ultrasonic image obtained in an analysis by the image analyzer; and
g) a secular change displayer for creating a graph illustrating a temporal change of the characteristic value and for displaying the graph on the monitor.

3. The ultrasonograph according to claim 2, wherein the characteristic value memory unit memorizes at least one of following values derived from the ultrasonic image: a main bang width, a stripe width, a number of stripes, and a sum value of a brightness in a brightest line and a sum value of a brightness in a darkest line.

4. The ultrasonograph according to claim 1, wherein the image analyzer obtains a width of a stripe appearing in the ultrasonic image due to a multiple reflection of an ultrasonic wave in the ultrasonic probe, and the determiner determines, based on the width of the stripe, a progression degree of an abrasion of an acoustic lens provided in the ultrasonic probe.

5. The ultrasonograph according to claim 1, wherein the image analyzer obtains a sum of brightness values in each line in the ultrasonic image, and the determiner determines whether or not the ultrasonic oscillator is damaged, based on the sum of the brightness values obtained for each line.

6. The ultrasonograph according to claim 1, wherein the image analyzer determines, in a case where a sum brightness for the entire ultrasonic image is equal to or less than a predetermined value, whether or not a brightness distribution of each line in the ultrasonic image satisfies a predetermined pattern, and the determiner determines a presence or absence of a detachment of an acoustic lens provided in the ultrasonic probe or a progression degree of the detachment, based on a presence or absence of a line satisfying the predetermined pattern or an occurrence ratio of such lines.

7. The ultrasonograph according to claim 2, wherein the image analyzer obtains a width of a stripe appearing in the ultrasonic image due to a multiple reflection of an ultrasonic wave in the ultrasonic probe, and the determiner determines, based on the width of the stripe, a progression degree of an abrasion of an acoustic lens provided in the ultrasonic probe.

8. The ultrasonograph according to claim 3, wherein the image analyzer obtains a width of a stripe appearing in the ultrasonic image due to a multiple reflection of an ultrasonic wave in the ultrasonic probe, and the determiner determines, based on the width of the stripe, a progression degree of an abrasion of an acoustic lens provided in the ultrasonic probe.

9. The ultrasonograph according to claim 2, wherein the image analyzer obtains a sum of brightness values in each line in the ultrasonic image, and the determiner determines whether or not the ultrasonic oscillator is damaged, based on the sum of the brightness values obtained for each line.

10. The ultrasonograph according to claim 3, wherein the image analyzer obtains a sum of brightness values in each line in the ultrasonic image, and the determiner determines whether or not the ultrasonic oscillator is damaged, based on the sum of the brightness values obtained for each line.

11. The ultrasonograph according to claim 2, wherein the image analyzer determines, in a case where a sum brightness for the entire ultrasonic image is equal to or less than a predetermined value, whether or not a brightness distribution of each line in the ultrasonic image satisfies a predetermined pattern, and the determiner determines a presence or absence of a detachment of an acoustic lens provided in the ultrasonic probe or a progression degree of the detachment, based on a presence or absence of a line satisfying the predetermined pattern or an occurrence ratio of such lines.

12. The ultrasonograph according to claim 3, wherein the image analyzer determines, in a case where a sum brightness for the entire ultrasonic image is equal to or less than a predetermined value, whether or not a brightness distribution of each line in the ultrasonic image satisfies a predetermined pattern, and the determiner determines a presence or absence of a detachment of an acoustic lens provided in the ultrasonic probe or a progression degree of the detachment, based on a presence or absence of a line satisfying the predetermined pattern or an occurrence ratio of such lines.
